(19) United States
(12) Patent Application Publication
(10) Pub. No.: US 2012/0191193 A1
(43) Pub. Date: Jul. 26, 2012

(54) INTERBODY IMPLANT SYSTEM AND METHODS OF USE

(75) Inventors: Hai H. Trieu, Cordova, TN (US); Cristian A. Capote, Memphis, TN (US); Jeffrey W. Beale, Bartlett, TN (US); Keith Miller, Germantown, TN (US)

(73) Assignee: Warsaw Orthopedic, Warsaw, IN (US)

(21) Appl. No.: 13/014,371

(22) Filed: Jan. 26, 2011

(51) Int. Cl. A61F 2/44

(52) U.S. Cl. 623/17.16

(57) ABSTRACT

An interbody implant spacer includes a flexible body defining a first surface configured for engagement with a first vertebral surface and a second surface configured for engagement with a second vertebral surface. The first surface includes at least one pre-formed protrusion extending outwardly therefrom. The body is expandable between a first, non-expanded configuration such that the at least one protrusion extends outwardly from the first surface and a second, expanded configuration such that the at least one protrusion extends outwardly from the first surface to engage the first vertebral surface and at least a portion of the second surface engages the second vertebral surface. Methods of use are disclosed.

### TECHNICAL FIELD

The present disclosure generally relates to medical devices, systems and methods for the treatment of musculoskeletal disorders, and more particularly to an interbody implant system and method that provides stabilization and height restoration for treating a vertebral column.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. After disc collapse, radiculopathy often causes severe pain and discomfort due to the pressure exerted on nerves and the spinal column.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Implantable prosthetics may employ interbody implants between vertebrae. This disclosure describes an improvement over these prior art technologies.

### SUMMARY OF THE INVENTION

Accordingly, an interbody implant system and method is provided that provides stabilization and height restoration for treating a vertebral column. It is contemplated that the interbody implant system includes an intervertebral spacer including an expandable chamber configured to define at least one protrusion. It is further contemplated that the interbody implant system and method may be employed for vertebral treatment.

In one embodiment, an interbody implant spacer is provided. The interbody implant spacer includes a flexible body defining a first surface configured for engagement with a first vertebral surface and a second surface configured for engagement with a second vertebral surface. The first surface includes at least one pre-formed protrusion extending outwardly therefrom. The body is expandable between a first, non-expanded configuration such that the at least one protrusion extends outwardly from the first surface and a second, expanded configuration such that the at least one protrusion extends outwardly from the first surface to engage the first vertebral surface and at least a portion of the second surface engages the second vertebral surface.

In one embodiment, the interbody implant spacer includes an inflatable body defining a first surface configured for engagement with a first vertebral surface and a second surface configured for engagement with a second vertebral surface. At least one preformed nipple extends outwardly from the first surface in a configuration to engage the first vertebral surface and gradually form an impression in the first vertebral surface. At least one pre-formed nipple extends outwardly from the second surface in a configuration to engage the second vertebral surface and gradually form an impression in the second vertebral surface. The body is expandable between a first, non-expanded configuration such that the at least one nipple of the first surface extends outwardly from the first surface and the at least one nipple of the second surface extends outwardly from the second surface, and a second, expanded configuration such that the at least one nipple of the first surface extends outwardly from the first surface in a configuration to engage the first vertebral surface and gradually form an impression in the first vertebral surface and the at least one nipple of the second surface extends outwardly from the second surface in a configuration to engage the second vertebral surface and gradually form an impression in the second vertebral surface.

In one embodiment, a method for treating vertebrae is provided. The method includes the steps of: making an incision in a body of a patient; creating a surgical pathway extending from the incision to an intervertebral disc space of the patient body; preparing the intervertebral disc space; providing an interbody implant spacer, similar to those described herein; delivering the spacer through the surgical pathway into the intervertebral disc space in a first, non-expanded configuration such that the at least one protrusion extends outwardly from the first surface; and expanding the spacer to a second, expanded configuration such that the at least one protrusion extends outwardly from the first surface to engage the first vertebral surface and at least a portion of the second surface engages the second vertebral surface.

### BRIEF DESCRIPTION OF THE DRAWINGS

The present disclosure will become more readily apparent from the specific description accompanied by the following drawings, in which:

FIG. 1 is a side view of one particular embodiment of an interbody implant spacer in accordance with the principles of the present disclosure;

FIG. 2 is a side view of the interbody implant spacer shown in FIG. 1;

FIG. 3 is a perspective view of one embodiment of the interbody implant spacer shown in FIG. 2;

FIG. 4 is a side view of one embodiment of the interbody implant spacer shown in FIG. 2;

FIG. 5 is a side view of vertebrae and a component of an interbody implant system in accordance with the principles of the present disclosure;

FIG. 6 is a side view of the vertebrae shown in FIG. 5 and components of the interbody implant system;

FIG. 7 is a side view of the vertebrae shown in FIG. 5, the interbody implant spacer shown in FIG. 1 and other components of the interbody implant system;

FIG. 8 is a side view of the vertebrae and the interbody implant spacer shown in FIG. 7;

FIG. 9 is a side view of one embodiment of the interbody implant spacer shown in FIG. 1; and

FIG. 10 is a side view of one embodiment of the interbody implant spacer shown in FIG. 2.

### DETAILED DESCRIPTION OF THE INVENTION

The exemplary embodiments of the interbody implant system and related methods of use disclosed are discussed in terms of medical devices for the treatment of musculoskeletal disorders and more particularly, in terms of an interbody implant that provides stabilization and height restoration for treating a vertebral column. It is envisioned that the interbody implant system provides a minimally invasive, low cost interbody stabilization device for patients with limited life expectancy and/or to treat patients with radiculopathy after disc collapse to provide height restoration between vertebral bodies. Such a configuration achieves anatomical distance between vertebrae while minimizing tissue dissection. It is further envisioned that the interbody implant can be in situ-formable with an inflatable chamber made of a textile material and inflatable via injection with an in situ curable polymer such as polymethyl methacrylate (PMMA) bone cement.

It is contemplated that the interbody implant has at least one protrusion for engagement with an upper endplate and/or a lower endplate. It is further contemplated that the radius of curvature of the protrusion is smaller than that of the endplates. The protrusion may be pre-formed or formed in situ. The protrusion may penetrate the tissues of the endplate immediately during implantation and/or following implantation. The protrusion and the resulting engagement with tissue resists migration while the remainder of the upper and/or lower surface of the interbody implant resists excessive subsidence with the tissues of the endplate to reduce damage to the endplates.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed interbody implant system may be alternatively employed in a surgical treatment with a patient in a prone or supine position, and/or employ various surgical approaches to the spine, including anterior, posterior, posterior mid-line, medial, lateral, postero-lateral, and/or antero-lateral approaches, and in other body regions. The present disclosure may also be alternatively employed with procedures for treating the lumbar, cervical, thoracic and pelvic regions of a spinal column. The interbody implant system and methods of the present disclosure may also be used on animals, bone models and other non-living substrates, such as, for example, in training, testing and demonstration.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

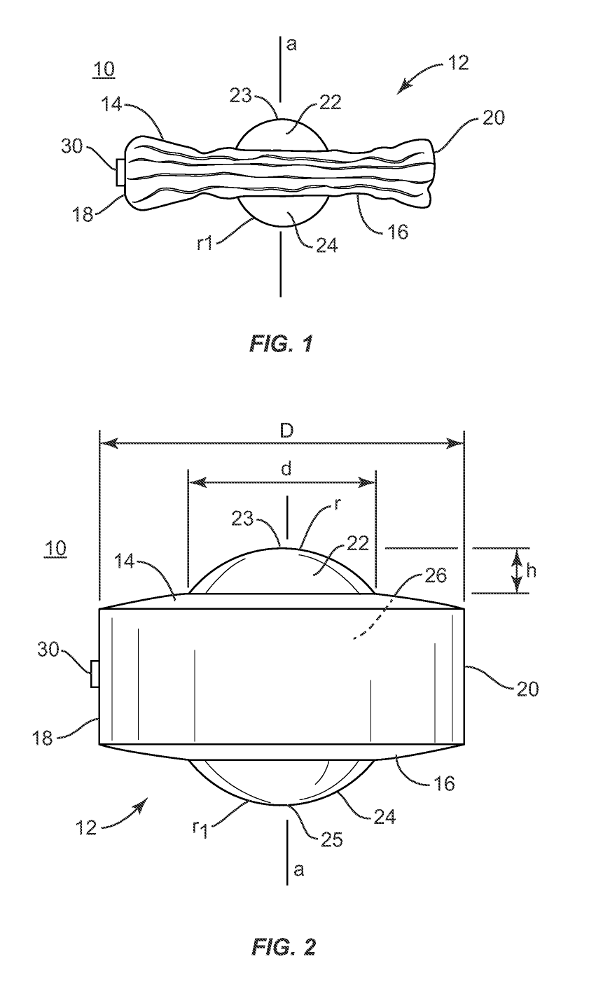

The following discussion includes a description of an interbody implant system and related methods of employing the interbody implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-2, there is illustrated components of an interbody implant system in accordance with the principles of the present disclosure.

The components of the interbody implant system can be fabricated from biocompatible materials suitable for medical applications, including metals, polymers, ceramics and/or their composites, depending on the particular application and/or preference of a medical practitioner. For example, the components of the interbody implant system, individually or collectively, can be fabricated from materials such as stainless steel, titanium, thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, and rigid polymers including polyphenylene, polyamide, polyimide, polyetherimide, polyethylene and epoxy. For example, an implant of the present disclosure may include a flexible body and a separate but integrated protrusion made from a relatively more rigid metal or plastic to penetrate and/or form an impression in tissue. Various components of the interbody implant system, may have material composites, including the above materials, to achieve various desired characteristics such as strength, rigidity, elasticity, compliance, biomechanical performance, durability and radiolucency or imaging preference.

The interbody implant system includes an interbody implant spacer 10 employed as a stabilization device in procedures, for example, for patients with limited life expectancy and/or to treat patients with radiculopathy after disc collapse to provide height restoration between vertebral bodies. Interbody implant spacer 10 achieves anatomical distance between vertebrae while minimizing tissue damage. The components of the interbody implant system may be monolithically formed, integrally connected or include fastening elements and/or instruments, as described herein.

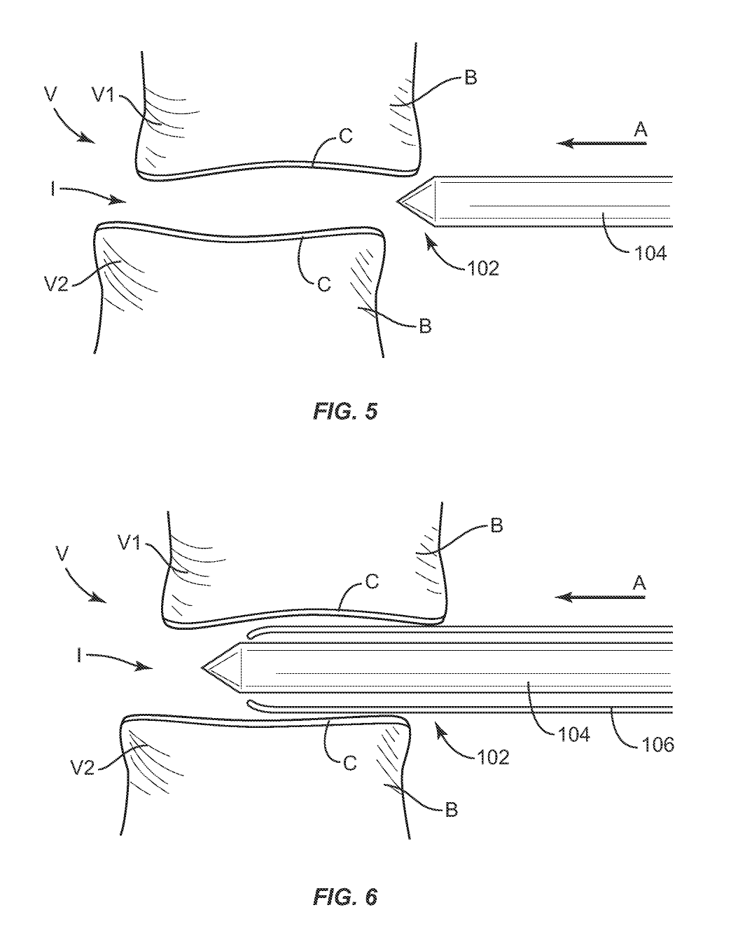

Interbody implant spacer 10 includes a flexible body, such as, for example, an inflatable body 12. Body 12 defines a first surface 14 configured for engagement with a first vertebral surface, such as, for example, an endplate of a first vertebrae V1 of vertebrae V (FIG. 5) and a second surface 16 configured for engagement with a second vertebral surface, such as, for example, an endplate of a second vertebrae V2. Body 12 also defines side surfaces 18, 20, which have a substantially arcuate configuration in the expanded configuration, discussed below. Surfaces 14, 16, 18, 20 have a substantially smooth configuration. It is envisioned that all or only a portion of each of surfaces 14, 16, 18, 20 may have alternate surface configurations, such as, for example, arcuate, undulating, rough, semi-porous, dimpled and/or textured. It is further envisioned that body 12 has an overall diameter D, in the expanded configuration discussed below, in the range of 8 millimeters (mm) to 32 mm, and preferably in the range of 12 mm and 28 mm.

First surface 14 includes a protrusion, such as, for example, a pre-formed nipple 22 extending outwardly from first surface 14. Nipple 22 is configured to engage the endplate of vertebrae V1, as will be described. Nipple 22 has a spherical configuration extending along a longitudinal axis a of body 12 and an arcuate distal tip 23. It is envisioned that nipple 22 may extend from body 12 in a rigid, semi-rigid or flexible configuration. It is further envisioned that nipple 22 may extend from first surface 14 a height h in the range of 1 mm to 7 mm and preferably in a range of 2 mm and 5 mm. It is contemplated that nipple 22 may be oriented, such as, for example, perpendicular, parallel, co-axial, angularly offset, offset and/or staggered relative to body 12. It is further contemplated that nipple 22 may extend from the outer surface of body 12 in a floppy configuration and/or inwardly until body 12 is expanded, as described below.

Nipple 22 has a substantially smooth surface. It is contemplated that nipple 22 may have a solid, hollow, porous or cage configuration. It is further contemplated that the cross-sectional geometry of nipple 22 along longitudinal axis a may have various configurations, for example, circular, oval, triangular, rectangular, polygonal, irregular, uniform, non-uniform, consistent or variable. It is envisioned that nipple 22 may have alternate surface configurations, such as, for example, those alternatives described herein. It is further envisioned that nipple 22 has a diameter d in the range of 4 mm to 16 mm and preferably in the range of 6 mm to 14 mm. It is further envisioned that nipple 22 has a maximum diameter that is approximately one half of the overall diameter of body 12, discussed above.

Nipple 22 has a spherical radius of curvature r, in the expanded configuration discussed below, for engaging the endplate of vertebrae V1. It is contemplated that nipple 22 has a radius r smaller than a radius of curvature of the endplate of vertebrae V1. It is further contemplated that nipple 22 has a radius r in the range of 2 mm to 20 mm and preferably in the range of 3 mm to 7 mm. It is envisioned that nipple 22 may be fabricated from the same or alternate material as body 12.

Second surface 16 includes a protrusion, such as, for example a pre-formed nipple 24 extending outwardly from second surface 16. Nipple 24 is configured to engage the endplate of vertebrae V2, as will be described. Nipple 24 has a spherical configuration extending along longitudinal axis a and an arcuate distal tip 25. It is envisioned that nipple 24 may extend from body 12 in a rigid, semi-rigid or flexible configuration. It is further envisioned that nipple 24 may extend from second surface 16 a height, similar to height h described above with regard to nipple 22, in the range of 1 mm to 7 mm and preferably in a range of 2 mm and 5 mm. It is contemplated that nipple 24 may be oriented, such as, for example, perpendicular, parallel, co-axial, angularly offset, offset and/or staggered relative to body 12 and/or nipple 22. It is further contemplated that nipple 24 may extend from the outer surface of body 12 in a floppy configuration and/or inwardly until body 12 is expanded, as described below.

Nipple 24 has a substantially smooth surface. It is contemplated that nipple 24 may have a solid, hollow, porous or cage configuration. It is further contemplated that the cross-sectional geometry of nipple 24 along longitudinal axis a may have alternate cross-section configurations, such as, for example, those alternatives described herein. It is envisioned that nipple 24 may have alternate surface configurations, such as, for example, those alternatives described herein. It is further envisioned that nipple 24 has a diameter, similar to diameter d described above with regard to nipple 22, in the range of 4 mm to 16 mm and preferably in the range of 6 mm to 14 mm. It is further envisioned that nipple 24 has a maximum diameter that is approximately one half of the overall diameter of body 12, discussed above.

Nipple 24 has a radius of curvature r1, in the expanded configuration discussed below, for engaging the endplate of vertebrae V2. It is contemplated that nipple 24 has a radius r1 smaller than a radius of curvature of the endplate of vertebrae V2. It is further contemplated that nipple 24 has a radius r1 in the range of 2 mm to 20 mm and preferably in the range of 3 mm to 7 mm. It is envisioned that nipple 24 may be fabricated from the same or alternate material as body 12 and/or nipple 22. It is further envisioned that nipple 22 may have the same or alternate spherical radius and/or height to nipple 24.

Body 12 is expandable from a first, non-expanded configuration (FIG. 1) such that nipple 22 extends outwardly from first surface 14 and nipple 24 extends outwardly from second surface 16, as described above. Body 12 defines a cavity, such as, for example, an inflatable chamber 26 configured for receiving a pressurized expanding medium to expand body 12 to a second, expanded configuration (FIG. 2). It is envisioned that body 12 may define one or a plurality of cavities configured for receiving a pressurized expanding medium, which may or may not be in communication and/or separately expandable. In one embodiment, body 12 includes two chambers (not shown) separated by a septum, which may be flexible, semi-rigid or rigid, such that each chamber is inflated via a separate port (not shown, similar to valve 30 described below) to a different pressure in a configuration to induce lordosis or correct a scoli.

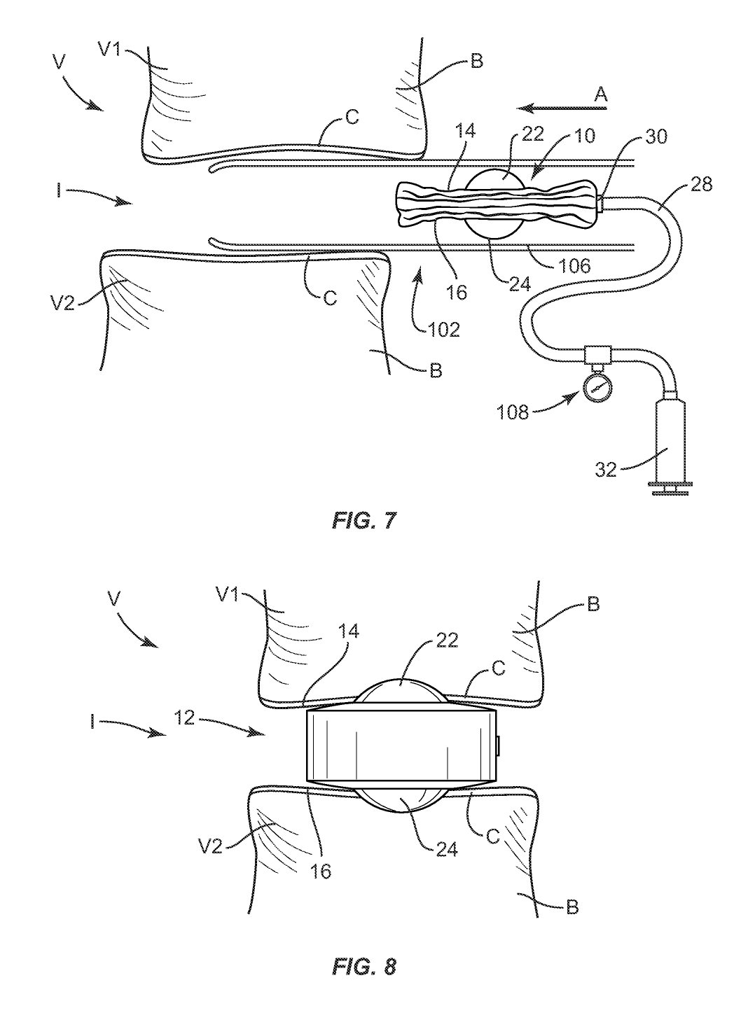

The interbody implant system includes an injection conduit, such as, for example, lumen 28 (FIG. 7) communicating with inflatable chamber 26 via a valve 30 of body 12. Lumen 28 is connected to a source 32 of pressurized expanding medium, such as, for example, inflating air, gas, fluid, and/or injectable polymer. Lumen 28 is configured to introduce the pressurized expanding medium from source 32 into inflatable chamber 26 to expand body 12 to the second, expanded configuration. It is contemplated that the pressurized expanding medium is introduced at a pressure in a range of 50 pounds per square inch (psi) to 800 psi and preferably in the range of 100 psi to 400 psi. The pressurized flow may be constant or varied, depending on the application. It is contemplated that alternative pressurized expanding mediums may be employed such as sterile water or saline. It is further contemplated that expansion of body 12 may be volume controlled. In one embodiment, a specific volume of PMMA bone cement is employed and injected into chamber 26 via valve 30 such that body 12 is expanded to the second, expanded configuration to a predetermined configuration and dimension. It is further contemplated that body 12 may be expanded with negative pressure. In one embodiment, body 12 is configured to vertically expand as side surfaces 18, 20 are caused to collapse internally, such as, for example by a vacuum, such that nipples 22, 24 expand to the second, expanded configuration.

Source 32 may be a syringe barrel with plunger, pressurized container and/or wall connection. The flow and/or pressure may be regulated and/or valve controlled manually, electronically or processor controlled, as is known to one skilled in the art. It is envisioned that body 12 and/or nipple 22 and/or nipple 24 may be fabricated from biologically acceptable materials including vinyl, polyvinyl chloride, silicone, nylon, thermoplastic rubbers, thermoplastic elastomer materials, polyethylenes, ionomer, polyurethane, polyolefins, polyetheretherketone, polyactide, polyglyclolide, poly(lactide-co-glycolide), poly(dioxanone), poly (ε-caprolactone), poly(hydroxylbutyrate), poly(hydroxylvalerate), tyrosine-based polycarbonate, polypropylene fumarate, polyethylene tetraphthalates (PET), or combinations thereof. Body 12 and/or nipple 22 and/or nipple 24 may be constructed of materials to achieve various desired characteristics such as biocompatibility, strength, thickness, rigidity, elasticity, durability, permeability. It is envisioned that body 12 in the first or second configuration may have various cross section configurations, such as, for example, those alternatives described herein.

It is contemplated that nipple 22 and/or nipple 24 may include radio opaque or radiolucent material for identification of depth within the endplate surfaces of vertebrae V1, V2. Nipples 22, 24 may include one or a plurality of guide marks. It is contemplated that the flexible body may include alternate or combinations of an expanding structure such as balloons, expanding arms, flexible wire, expanding linkages, tongs, expanding bands and articulating linkages.

In the second, expanded configuration, nipple 22 extends outwardly from first surface 14 in a configuration to engage the endplate of vertebrae V1 and gradually form an impression in the endplate of vertebrae V1. Nipple 24 extends outwardly from second surface 16 in a configuration to engage the endplate of vertebrae V2 and gradually form an impression in the endplate of vertebrae V2. The configuration of nipples 22, 24 provide a selective subsidence to form the impression in the tissues of the endplates of vertebrae V1, V2. It is contemplated that the tissue includes bone, cortical bone, cancellous bone, cartilage, connective tissue, muscle, membrane and combinations thereof.

For example, upon disposal of body 12 with an intervertebral disc space I (FIG. 8), nipples 22, 24 immediately create an impression and penetrate an outer cartilage surface C of the endplates of vertebrae V1, V2. Over time, nipples 22, 24 gradually create an impression in bone B, which includes cortical bone and/or cancellous bone, of the endplates of vertebrae V1, V2. The remainder of surfaces 14, 16 engage the tissues of the endplates of vertebrae V1, V2 to provide distraction and load support. As nipples 22, 24 are caused to engage bone B, bone B gradually over time deforms and subsides about nipples 22, 24 to anchor body 12 in intervertebral disc space I. This selective subsidence gradually creates the impression within the endplates of vertebrae V1, V2 adjacent nipples 22, 24 to equalize the contact stress with body 12 over a greater surface area. This configuration resists migration of interbody implant spacer 10 within intervertebral disc space I with minimal damage to the tissues of the endplates of vertebrae V1, V2. In one embodiment, the tissues of the endplates of vertebrae V1, V2 do not include cartilage, for example, due to preparation of the surfaces of vertebrae V1, V2, and nipples 22, 24 create an impression and penetrate bone B only.

It is contemplated that the selective subsidence, for example, the depth of engagement, impression and/or penetration, can be controlled or regulated, by various elements, such as, for example, expansion parameters of body 12 including pressure, materials employed, hardness and/or density of tissues. In one embodiment, the protrusion is configured to create an impression and/or penetrate only cartilage surface C. In one embodiment, the protrusion has a hardness substantially equivalent or greater than cartilage surface C. It is envisioned that the protrusion may create an impression and/or penetrate one or more tissues of the endplates of vertebrae V1, V2 immediately or gradually over time in a selected subsidence. It is contemplated that the protrusion creates an impression, including indentation, penetration, piercing and contacting, non-penetration up to a predetermined threshold of depth. It is further contemplated that the that the selective subsidence may be in a range of 1 mm to 7 mm and preferably in a range of 2 mm and 5 mm.

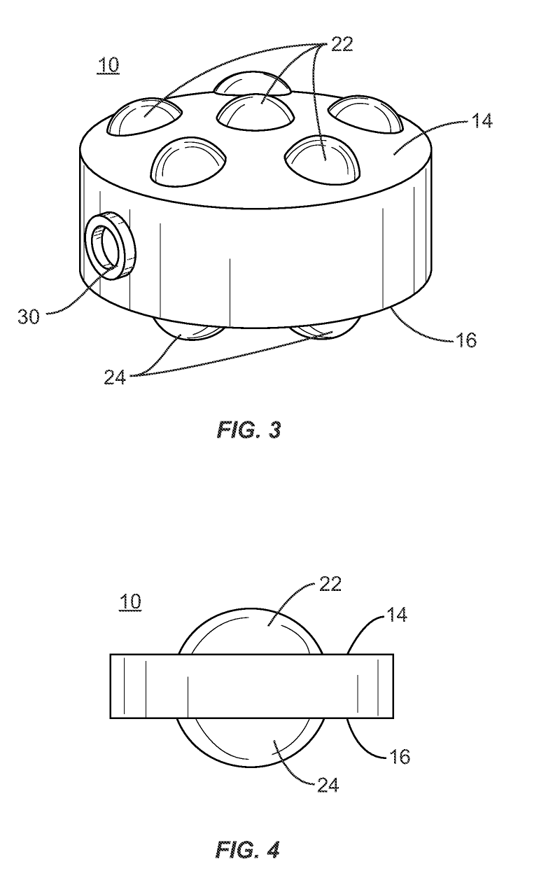

In one embodiment of interbody implant spacer 10, as shown in FIG. 3, surface 14 includes a plurality of nipples 22 and surface 16 includes a plurality of nipples 24. It is contemplated that nipple(s) 22 may have various cross section geometry, material and orientation configurations relative to other nipples 22, 24, and nipple(s) 24 may have various cross section geometry, material and orientation configurations relative to other nipples 22, 24. It is further contemplated one or a plurality of protrusions may be employed with surfaces 14, 16. In one embodiment of interbody implant spacer 10, as shown in FIG. 4, surfaces 14, 16 are substantially even or planar relative to the curvature of nipples 22, 24, respectively.

In assembly, operation and use, the interbody implant system is employed with a surgical procedure, such as, a treatment of a spine of a patient including vertebrae V, intervertebral disc space I and body areas adjacent thereto, as discussed herein. The interbody implant system may be employed with surgical procedures, such as, for example, discectomy, laminotomy, laminectomy, nerve root retraction, foramenotomy, facetectomy, decompression, spinal nucleus or disc replacement.

For example, the interbody implant system can be employed with a surgical procedure, such as, for example, an interbody stabilization, for patients with limited life expectancy and/or to treat patients with radiculopathy after disc collapse to provide height restoration between vertebral bodies, of an applicable condition or injury of an affected section of a spinal column and adjacent areas within a body, such as, for example, intervertebral disc space I between the endplate of vertebrae V1 and the endplate of vertebrae V2 of vertebrae V. It is contemplated that interbody implant spacer 10 of the interbody implant system can be inserted with intervertebral disc space I to space apart articular joint surfaces, provide support and maximize stabilization of vertebrae V.

In use, as shown in FIGS. 5-8, to treat the affected section of vertebrae V, a medical practitioner obtains access to a surgical site including vertebrae V in any appropriate manner, such as through incision and retraction of tissues. It is envisioned that interbody implant spacer 10 can be used in any existing surgical method or technique including open surgery, mini-open surgery, minimally invasive surgery and percutaneous surgical implantation, whereby vertebrae V is accessed through a mini-incision, or sleeve that provides a protected passageway to the area. Once access to the surgical site is obtained, the particular surgical procedure is performed for treating the spine disorder. Interbody implant spacer 10 is then employed to augment the surgical treatment. Interbody implant spacer 10 can be completely or partially revised, removed or replaced in situ. It is contemplated that one or all of the components of the interbody implant system can be delivered to the surgical site via manual manipulation and/or a free hand technique. It is further contemplated that interbody implant spacer 10 may be inserted posteriorly, and then manipulated anteriorly and/or lateral and/or medial.

An incision is made in the body of a patient and a cutting instrument (not shown) creates a surgical pathway 102 for implantation of interbody implant spacer 10 within the patient body. A guide instrument 104 is employed to initially distract vertebrae V1 from vertebrae V2, as manipulated in the direction of arrow A shown in FIG. 5. A sleeve or cannula 106 is used to access intervertebral disc space I, as manipulated in the direction of arrow A shown in FIG. 6, and facilitate delivery and access for components of the interbody implant system. A preparation instrument (not shown) can be inserted within cannula 106 and disposed within intervertebral disc space I. The preparation instrument(s) can be employed to remove some or all of the disc tissue including the disc nucleus and fluids, adjacent tissues and/or bone, corticate, scrape and/or remove tissue from the surfaces of endplates of opposing vertebrae V1, V2, as well as for aspiration and irrigation of the region according to the requirements of a particular surgical application.

Interbody implant spacer 10, with body 12 disposed in the first, non-expanded configuration, is delivered through surgical pathway 102 into intervertebral disc space I with a delivery instrument (not shown) including a driver (not shown) via sleeve 106, as manipulated in the direction of arrow A shown in FIG. 7. The driver delivers interbody implant spacer 10 into the prepared intervertebral disc space I, between vertebrae V1 and vertebrae V2, according to the requirements of a particular surgical application. Interbody implant spacer 10 is manipulated such that opposing surfaces 14, 16 of body 12 will engage endplates of opposing vertebrae V1, V2 upon inflation of body 12.

Lumen 28 communicates with inflatable chamber 26 via valve 30, as described above. Lumen 28 is connected to source 32, as regulated by gauge 108, which supplies pressurized expanding medium into inflatable chamber 26 to expand body 12 to the second, expanded configuration. According to the particular surgical application, gauge 108 is set to a particular pressure, similar to those described above, chamber 26 is filled with the pressurized expanding medium at that pressure and body 12 is inflated.

In the second, expanded configuration, nipple 22 extends outwardly from first surface 14 in a configuration to engage the endplate of vertebrae V1. Nipple 24 extends outwardly from second surface 16 in a configuration to engage the endplate of vertebrae V2. The configuration of nipples 22, 24 cause a selective subsidence in the surrounding tissues such that an impression forms in the tissues of the endplates of vertebrae V1, V2.

Upon disposal of body 12 with intervertebral disc space I, nipples 22, 24 immediately create an impression and penetrate outer cartilage surface C of the endplates of vertebrae V1, V2. Over time, nipples 22, 24 gradually create an impression in bone B of the endplates of vertebrae V1, V2, as shown in FIG. 8. The remainder of surfaces 14, 16 engage the tissues of the endplates of vertebrae V1, V2 to provide distraction and load support. As nipples 22, 24 are caused to engage bone B, bone B gradually over time deforms and subsides about nipples 22, 24 to anchor body 12 in intervertebral disc space I. This selective subsidence gradually creates the impression or indentation within the endplates of vertebrae V1, V2 adjacent nipples 22, 24 to equalize the contact stress with body 12 over a greater surface area. This configuration resists migration of interbody implant spacer 10 within intervertebral disc space I with minimal damage to the tissues of the endplates of vertebrae V1, V2.

The components of the interbody implant system secure and stabilise vertebrae V in connection with the surgical implant procedure while preventing undesired migration of body 12. It is envisioned that one or a plurality of interbody implant spacers 10 may be used for a surgical procedure employing the interbody implant system.

In one embodiment, the interbody implant system includes bone growth promoting material, which may be disposed, packed or layered within, on or about the components and/or surfaces thereof. The bone growth promoting material, such as, for example, bone graft can be a particulate material, which may include an osteoconductive material such as hydroxyapatite and/or an osteoinductive agent such as a bone morphogenic protein to enhance bony fixation of interbody implant spacer 10 with the adjacent vertebrae V.

Interbody implant spacer 10 may include bone growth promoting material, which may be disposed, packed or layered within, on or about the surfaces of body 12. The bone growth promoting material, such as, for example, bone graft, is configured for disposal within, about and/or adjacent surfaces of vertebrae V1, V2.

It is envisioned that the bone graft is a particulate material, which may include an osteoconductive material such as hydroxyapatite and/or an osteoinductive agent such as a bone morphogenic protein (BMP) to enhance bony fixation of body 12 with the adjacent vertebrae V.

It is contemplated that the bone graft may include therapeutic polynucleotides or polypeptides. It is further contemplated that the bone graft may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, Growth and Differentiation Factors proteins (GDF) and cytokines. Interbody implant spacer 10 can be made of radiolucent materials such as polymers. Radiomarkers may be included for identification under x-ray, fluoroscopy, CT or other imaging techniques.

In one embodiment, interbody implant system may include at least one agent including biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, biologically active agents coated onto the exterior of body 12 and/or applied thereto for gradual release such as by blending in a bioresorbable polymer that releases the biologically active agent or agents in an appropriate time dependent fashion as the polymer degrades within the patient. Suitable biologically active agents include, for example, BMP and cytokines.

It is envisioned that the agent may include one or a plurality of therapeutic agents and/or pharmacological agents for release, including sustained release, to treat, for example, pain, inflammation and degeneration. The agents may include pharmacological agents, such as, for example, antibiotics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof.

The agent may also include analgesics or anesthetics such as acetic acid derivatives, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamthoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

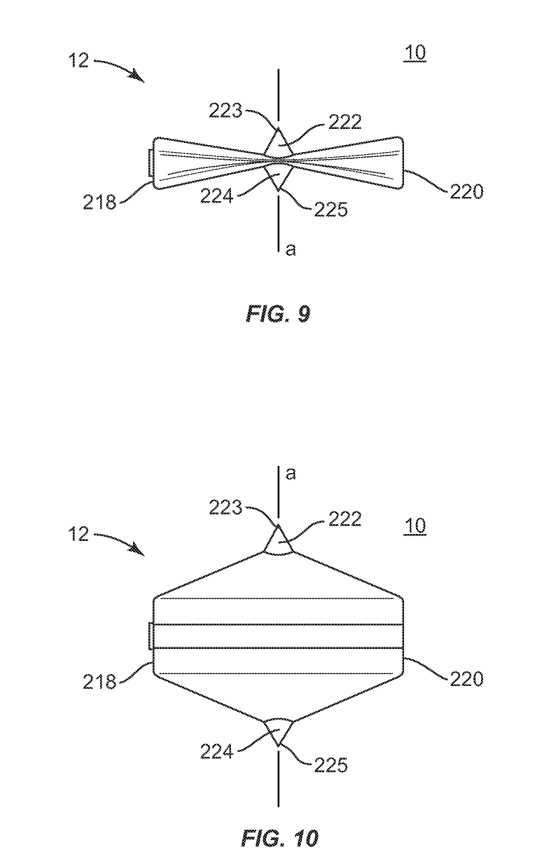

In one embodiment, as shown in FIGS. 9-10, interbody implant spacer 10, similar to that described above, includes body 12 having substantially linear side surfaces 218, 220. Body 12 includes a nipple 222, similar to nipple 22 described above, having a conical configuration extending along longitudinal axis a and a pointed distal tip 223. Body 12 also includes a nipple 224, similar to nipple 24 described above, having a conical configuration extending along longitudinal axis a and a pointed distal tip 225. Body 12 is expandable between a first, non-expanded configuration (FIG. 9) and a second, expanded configuration (FIG. 10), similar to that described above with regard to FIGS. 1-2. It is contemplated that the components of body 12 may be fabricated from the materials described herein, and/or a superelastic metallic alloys (e.g. Nitinol, super elasto-plastic metals, such as GUM METAL® manufactured by Toyota Material Incorporated of Japan). In one embodiment, nipples 222, 224 each have a conical configuration and a blunt tip.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An interbody implant spacer, comprising:
a flexible body defining a first surface configured for engagement with a first vertebral surface and a second surface configured for engagement with a second vertebral surface, the first surface including at least one pre-formed protrusion extending outwardly therefrom,
wherein the body is expandable between a first, non-expanded configuration such that the at least one protrusion extends outwardly from the first surface and a second, expanded configuration such that the at least one protrusion extends outwardly from the first surface to engage the first vertebral surface and at least a portion of the second surface engages the second vertebral surface.

2. The interbody implant spacer according to claim 1, wherein the second surface includes at least one pre-formed protrusion extending outwardly therefrom such that in the first, non-expanded configuration the at least one protrusion of the second surface extends outwardly from the second surface and in the second, expanded configuration the at least one protrusion of the second surface extends outwardly from the second surface to engage the second vertebral surface.

3. The interbody implant spacer according to claim 1, wherein the at least one protrusion is configured to engage the first vertebral surface and gradually form an impression in the first vertebral surface.

4. The interbody implant spacer according to claim 1, wherein the at least one protrusion is configured to engage the first vertebral surface in a selective subsidence to form an impression in the first vertebral surface to a predetermined threshold.

5. The interbody implant spacer according to claim 1, wherein the first vertebral surface includes an outer cartilage surface such that the at least one protrusion is configured to penetrate only the outer cartilage surface.

6. The interbody implant spacer according to claim 1, wherein the at least one protrusion has a hardness that is greater than a hardness of an outer cartilage surface of the first vertebral surface.

7. The interbody implant spacer according to claim 1, wherein the at least one protrusion is configured to penetrate an outer cartilage surface of the first vertebral surface immediately and gradually form an impression in cancellous bone of the first vertebral surface.

8. The interbody implant spacer according to claim 1, wherein the expandable body includes an inflatable member.

9. The interbody implant spacer according to claim 8, wherein the inflatable member is inflated with a curable polymer.

10. The interbody implant spacer according to claim 1, wherein the at least one protrusion defines a first radius of curvature and the first vertebral surface defines a second radius of curvature that is greater than the first radius of curvature.

11. The interbody implant spacer according to claim 1, wherein the at least one protrusion has an arcuate configuration.

12. The interbody implant spacer according to claim 1, wherein the at least one protrusion defines a pointed distal tip.

13. The interbody implant spacer according to claim 1, wherein the at least one protrusion includes a plurality of pre-formed protrusions extending outwardly from the first surface of the expandable body.

14. An interbody implant spacer, comprising:
an inflatable body defining a first surface configured for engagement with a first vertebral surface and a second surface configured for engagement with a second vertebral surface, at least one pre-formed nipple extending outwardly from the first surface in a configuration to engage the first vertebral surface and gradually form an impression in the first vertebral surface and at least one pre-formed nipple extending outwardly from the second surface in a configuration to engage the second vertebral surface and gradually form an impression in the second vertebral surface,
wherein the body is expandable between a first, non-expanded configuration such that the at least one nipple of the first surface extends outwardly from the first surface and the at least one nipple of the second surface extends outwardly from the second surface, and a second, expanded configuration such that the at least one nipple of the first surface extends outwardly from the first surface in a configuration to engage the first vertebral surface and gradually form an impression in the first vertebral surface and the at least one nipple of the second surface extends outwardly from the second surface in a configuration to engage the second vertebral surface and gradually form an impression in the second vertebral surface.

15. The interbody implant spacer according to claim 14, wherein the at least one nipple of the first surface is configured to engage the first vertebral surface in a selective subsidence to form an impression in the first vertebral surface to a predetermined threshold and the at least one nipple of the second surface is configured to engage the second vertebral surface in a selective subsidence to form an impression in the second vertebral surface to a predetermined threshold.

16. The interbody implant spacer according to claim 14, wherein the at least one nipple of the first surface has a hardness that is greater than a hardness of an outer cartilage surface of the first vertebral surface such that the at least one nipple of the first surface is configured to penetrate the outer cartilage surface of the first vertebral surface immediately and gradually form an impression in cancellous bone of the first vertebral surface, and the at least one nipple of the second surface has a hardness that is greater than a hardness of an outer cartilage surface of the second vertebral surface such that the at least one nipple of the second surface is configured to penetrate the outer cartilage surface of the second vertebral surface immediately and gradually form an impression in cancellous bone of the second vertebral surface.

17. A method for treating vertebrae, the method comprising the steps of:
making an incision in a body of a patient;
creating a surgical pathway extending from the incision to an intervertebral disc space of the patient body;
preparing the intervertebral disc space;
providing an interbody implant spacer, the spacer including a flexible body defining a first surface configured for engagement with a first vertebral surface and a second surface configured for engagement with a second vertebral surface, the first surface including at least one pre-formed protrusion extending outwardly therefrom;
delivering the spacer through the surgical pathway into the intervertebral disc space in a first, non-expanded configuration such that the at least one protrusion extends outwardly from the first surface; and
expanding the spacer to a second, expanded configuration such that the at least one protrusion extends outwardly from the first surface to engage the first vertebral surface and at least a portion of the second surface engages the second vertebral surface.

18. The method according to claim 17, further comprising the step of disposing the at least one protrusion into engagement with the first vertebral surface such that the at least one protrusion gradually forms an impression in the first vertebral surface.

19. The method according to claim 17, further comprising the step of disposing the at least one protrusion into engagement with the first vertebral surface in a selective subsidence to form an impression in the first vertebral surface to a predetermined threshold.

20. The method according to claim 17, further comprising the step of disposing the at least one protrusion into engagement with the first vertebral surface such that the at least one protrusion is configured to penetrate an outer cartilage surface of the first vertebral surface immediately and gradually form an impression in cancellous bone of the first vertebral surface.
